(19) United States
(12) Patent Application Publication
(10) Pub. No.: US 2026/0069302 A1
(43) Pub. Date: Mar. 12, 2026

(54) VARIABLE MAXIMUM FORCE LAPAROSCOPIC SEALER AND DIVIDER

(71) Applicant: GYRUS ACMI, INC. D/B/A OLYMPUS SURGICAL TECHNOLOGIES AMERICA, Westborough, MA (US)

(72) Inventors: Charles E. Wood, Eden Prairie, MN (US); Theodore C. Blus, Arden Hills, MN (US); William E. Butler, Minneapolis, MN (US)

(21) Appl. No.: 19/389,531

(22) Filed: Nov. 14, 2025

Related U.S. Application Data
(63) Continuation of application No. PCT/US2024/029774, filed on May 16, 2024.
(60) Provisional application No. 63/466,809, filed on May 16, 2023.

(51) Int. Cl. A61B 17/28

(52) U.S. Cl. CPC A61B 17/28

(57) ABSTRACT

A medical device such as a surgical forceps is usable with at least two different jaw forces. The device can include a longitudinal shaft, having a proximal portion and a distal portion. An end effector can be attached to and can extend from the distal portion. A compressible member can be aligned with the longitudinal shaft. The compressible member can be configured for applying a variable maximum bias force for communication to the end effector. An end-user-positionable seat can be located against a first end of the compressible member. The seat can be actuatable by the end-user for varying the variable maximum bias force.

### CROSS-REFERENCE TO RELATED APPLICATIONS

This application is a Continuation of International Patent Application No. PCT/US2024/029774 entitled “VARIABLE MAXIMUM FORCE LAPAROSCOPIC SEALER AND DIVIDER,” filed May 16, 2024, which claims the benefit of priority to U.S. Provisional Patent Application Ser. No. 63/466,809 entitled “VARIABLE MAXIMUM FORCE LAPAROSCOPIC SEALER AND DIVIDER,” filed May 16, 2023, the disclosures of which are incorporated herein in their entireties by reference.

### TECHNICAL FIELD

This document pertains generally, but not by way of limitation, to systems and methods for actuating end effectors of medical devices. In particular, the systems and methods can be used in or with a forceps having an actuatable jaw and/or a blade.

### BACKGROUND

Medical devices for diagnosis and treatment, such as a forceps, are used for medical procedures such as laparoscopic and open surgeries. Forceps can be used to manipulate, engage, grasp, or otherwise affect an anatomical feature, such as a vessel or other tissue. Such medical devices can include an end effector that is one or more of: rotatable, openable, closeable, extendable, retractable, and capable of supplying an input such as electromagnetic energy or ultrasound.

For example, jaws located at a distal end of a forceps can be actuated via elements at a handpiece of the forceps to cause the jaws to open and close and thereby engage the vessel or other tissue. Forceps may also include an extendable and retractable blade, such as a blade that can be extended longitudinally between a pair of jaws.

### SUMMARY OF THE DISCLOSURE

A medical device can include a longitudinal shaft. The shaft can have a proximal portion and a distal portion. An end effector can be attached to and can extend from the distal portion. A compressible member can be aligned with the longitudinal shaft. The compressible member can be configured for applying a variable maximum bias force for communication to the end effector. An end-user-positionable seat can be located against a first end of the compressible member. The seat can be actuatable by an end-user, such as for varying the variable maximum bias force.

The medical device can include a longitudinal shaft, having a proximal portion and a distal portion. An end effector can be attached to and can extend from the distal portion. A handpiece can be operably connected to the end effector. The handpiece can be attached to and can extend from the proximal portion of the longitudinal shaft. A variable motion transfer assembly can be actuatable, such as for adjusting force effected by the end effector.

In use, a surgical method can include applying a first force to tissue with a surgical forceps being in a first mode of operation. The method can include switching the surgical forceps from the first mode of operation to a second mode of operation. The second mode of operation can comprise moving a user-positionable seat, such as to compress a compressible member within the forceps such that the surgical forceps are actuatable for applying a second force that is greater than the first force.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a side view of a forceps showing jaws in an open position.

FIG. 1B illustrates a side view of the forceps of FIG. 1A showing the jaws in a closed position.

FIG. 1C illustrates a partial cross-sectional view of the forceps of FIG. 1A showing the lever moved further proximally (e.g., a force limiting state, an over-travel position).

FIGS. 2A-2C illustrate views of a forceps having a variable motion transfer assembly.

FIGS. 3A-3C illustrate views of a forceps having a variable motion transfer assembly.

FIG. 4 illustrates a method of using a forceps having a variable motion transfer assembly.

FIG. 5 depicts a device with a sensor design for measuring jaw force settings in an example.

FIGS. 6A-6B depict an example a jaw force device with a strain gauge in an example.

FIG. 7 depicts an additional example of a strain gauge.

FIGS. 8A-8B depict examples of such Wheatstone bridges where the resistors are strain gauges.

FIGS. 9A-9E depict an example of a device with a strain gauge.

FIG. 10 depicts a method of sensing and communicating jaw force level or state.

FIG. 11 is a block diagram illustrating an example of a machine upon which one or more embodiments may be implemented.

### DETAILED DESCRIPTION

Discussed herein is a surgical device with an end-user-adjustable maximum jaw force. The device can be used, for example, in laparoscopic procedures, such as for sealing and dividing vessels. The device can include a medical device with a jaw, such as a forceps. This can be compared with an approach that only allows for a specific maximum force to be applied by the jaws on a surgical site, such as to seal a vessel between the jaws. During surgery, a surgeon (or other end-user) may run across multiple vessels or surgical sites that can benefit from different ranges of force to be applied by the jaw of the medical device. In a fixed-maximum-force approach, while the end-user could vary the applied force by squeezing harder on a lever at the forceps handle, that variability is limited by a specific maximum-force established at manufacture. Beyond that manufactured maximum-force, the surgeon or other end-user likely had to either switch devices, or was not able to as effectively seal a particular vessel, such as a vessel larger than 7 mm, without a clip or other component being brought into the surgical site. Where the device's maximum force can be changed by the surgeon or other end-user, more flexibility is given with a particular device in this situation.

Here, the medical device can be provided with the internal ability to alter or change the maximum force appliable by the jaws by changing the pressure exerted on the jaws. This can be accomplished through a motion transfer assembly in the proximal body of the device. A compressible member (e.g., a spring) aligned within the shaft can be adjusted by the user to adjust force exerted by the jaws. The compressible member can be adjusted by a rotating or other variable-positioning component, such as a hex nut, which can be laterally moved along the shaft to change the amount of biasing provided by the compressible member. An end-user-positionable seat can be used to secure the compressible member and fasten the device at a desired maximum force amount.

Adjusting the compressible member can allow the user can change the maximum bias force allowed in the device. The compressible member, when adjusted to a desired compression, applies a constant force per unit distance. This force changes depending on the amount of compression applied to the compressible member. The more compressed the compressible member, the greater the force. Thus, when a user adjusts the compressible member, and secures the compressible member in a new compression state (such as with the end-user positionable seat), a different maximum bias force can be applied with the device.

For example, a forceps medical device can include a handpiece that allows a surgeon (or other end-user) to control an end effector on that device, such as jaws or a blade. Actuation of the end effector can be affected through one or more actuation systems of the handpiece, such as to allow the surgeon to retract, extend, or rotate one or more shafts and control actions of the end effector. In other variations, the medical device can include a different or additional end effector, handpiece, or both.

A surgeon may use such a forceps medical device for sealing vessels, such as during a laparoscopic procedure. In an approach, a forceps can be made for sealing of vessels of particular sizes. For example, a surgeon may begin a procedure with a particular forceps that is configured to provide appropriate force for sealing vessels of less than 7 mm in diameter. However, if the surgeon comes across a vessel needing sealing and that vessel is larger than 7 mm in diameter, such as about 8 mm or 10 mm, the surgeon may accommodate by switching instruments. This can be done, for example, by using a laparoscopic clip in addition to the forceps. However, using such clips can include changing instruments from the forceps to a clip applicator, increasing surgery duration. For this reason, a forceps device that can be used for both sealing of smaller vessels and sealing of larger vessels would reduce the need for switching devices and allow for more efficient procedures when larger vessels are found.

Other approaches may limit such medical devices in that the maximum force that can be provided by the forceps is constrained to a specific amount; such an approach does not provide an end-user with an ability to alter or change the maximum amount of force being applied by the forceps. In other words, such an approach is limited to a single maximum jaw force. Three parameters control vessel sealing ability: energy, time, and force. A device with an end-user-specifiable variable maximum jaw force, whether discrete or continuous, can help optimize generator energy, surgery time, or both.

Thus, discussed herein is a medical device forceps with an end-user specifiable variable maximum jaw force provided by a variable motion transfer assembly. Such a device can help improve vessel seal burst pressure performance. For example, a lower jaw force can be used for small to medium size vessels, while a higher jaw force can be used on large diameter vessels. This can allow for seal optimization across different vessel sizes. Moreover, higher jaw force can help reduce or minimize thermal spread during hemostasis, and can help when grasping and sealing a tissue bundle. Such variable force can be used to effectively exert force along the jaw length to provide a consistent seal, without need for additional dissection or isolation. However, higher jaw force can sometimes produce sticking, making the ability to switch between lower and high jaw force beneficial overall. Additionally, the forceps with variable maximum force discussed herein can include a handpiece and end effector that allow for improvements such as reduced packing space, a simplified design and manufacturing, improved user experience, and increased stability, preventing damage to the forceps themselves.

Such forceps can include a medical forceps, a cutting forceps, an electrosurgical forceps, or any other type of forceps. The forceps can include an end effector that is controlled by a handpiece including an actuation system to be one or more of: rotatable, openable, closeable, extendable, and capable of supplying electromagnetic or acoustic energy. For example, jaws located at a distal end of the forceps can be actuated via one or more actuators at a handpiece of the forceps to cause the jaws to open, close and rotate to engage a vessel or other tissue. The forceps may also include an extendable and retractable blade, such as blades that can be extended longitudinally in between a pair of jaws to separate a first tissue from a second tissue.

Although the present application is described with reference to a forceps, other end effectors can be used with and operated by the handpiece described herein. In addition, other handpieces can be connected to and can control the end effectors described herein. This disclosure includes examples of handpieces including one or more actuation systems, examples of end effectors, and examples where the disclosed actuation systems and end effectors can be used together in a medical device. Examples of illustrative devices and forceps are shown and discussed in U.S. 2020/0305960, which is herein incorporated by reference in its entirety.

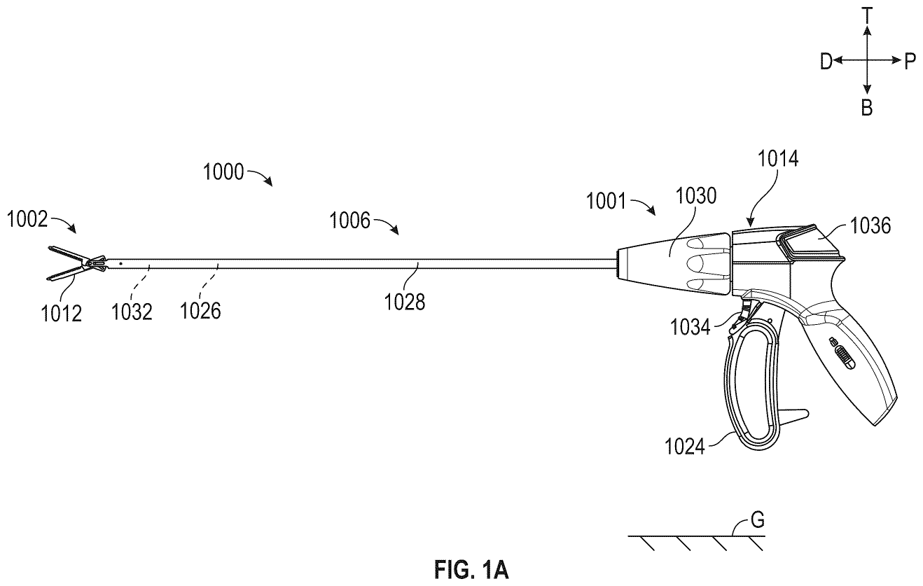

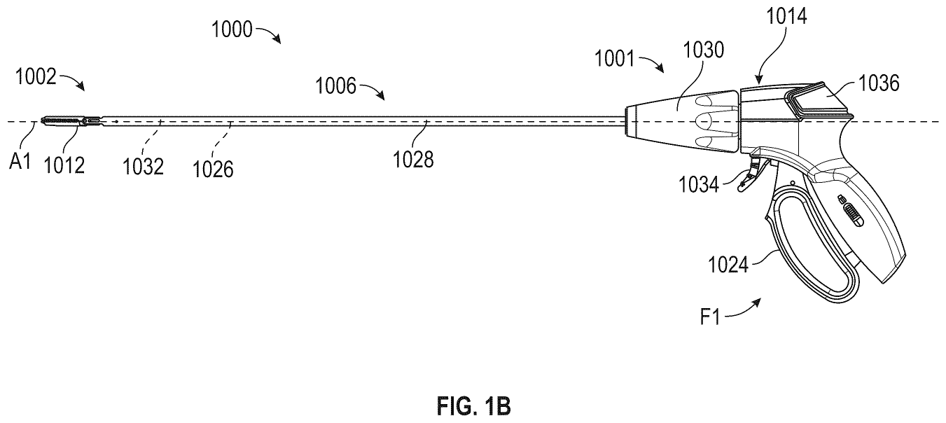

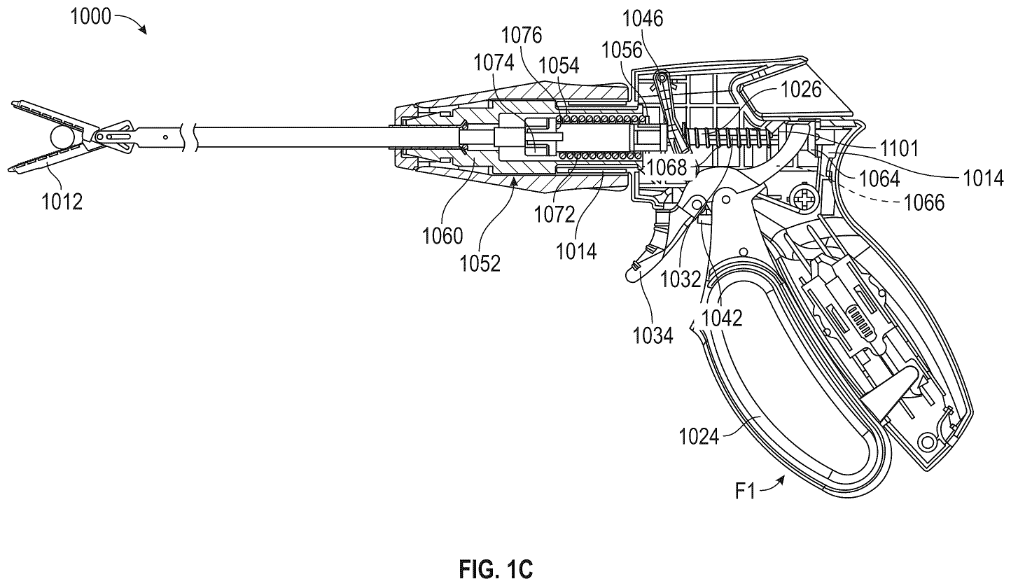

FIGS. 1A to 1C illustrate an example of a forceps with a motion transfer assembly in various positions. FIG. 1A illustrates a side view of a forceps showing jaws in an open position. FIG. 1B illustrates a side view of the forceps of FIG. 1A showing the jaws in a closed position. FIG. 1C depicts a cross-sectional view of the forceps 1000 showing the motion transfer assembly.

FIG. 1A illustrates a side view of a forceps 1000 with jaws 1012 in an open position. FIG. 1B illustrates a side view of the forceps 1000 with the jaws 1012 in a closed position. Directional descriptors such as proximal and distal are used within their ordinary meaning in the art. The proximal direction P and distal direction D are indicated on the axes provided in FIG. 1A.

The forceps 1000 can include a handpiece 1001 at a proximal portion, and an end effector 1002 at a distal portion. An intermediate portion 1006 can extend between the handpiece 1001 and the end effector 1002 to operably couple the handpiece 1001 to the end effector 1002. Various movements of the end effector 1002 can be controlled by one or more actuation systems of the handpiece 1001, such as by the motion transfer assembly. The end effector 1002 can include jaws 1012 that are capable of opening and closing, such as for grasping and sealing vessels. The end effector 1002 can be rotated about a longitudinal axis A1 (FIG. 1B) of the forceps 1000. In some cases, the end effector 1002 can include a cutting blade, and an electrode for applying electromagnetic energy.

With reference to FIGS. 1A and 1, and overview of the forceps 1000 is shown and discussed. Here, two motion transfer assemblies can provide transmission of forces received from a user, via clamping (e.g., via a lever 1024) and a rotational actuator 1030, to the jaws 1012 of the forceps 1000 to actuate clamping and rotation of the jaws 1012.

As shown in FIGS. 1A and 1, the forceps 1000 can include the jaws 1012, a housing 1014, a lever 1024, a drive shaft 1026, an outer shaft 1028, a rotational actuator 1030, a blade assembly, a trigger 1034 and an activation button 1036. In this example, the end effector 1002, or a portion of the end effector 1002 can be one or more of: opened, closed, rotated, extended, retracted, and electromagnetically energized such as with radiofrequency energy.

To operate the end effector 1002, the user can displace the lever 1024 proximally by applying Force F1 (FIG. 1B) to drive the jaws 1012 from the open position (FIG. 1A) to the closed position (FIG. 1), thereby providing force to the jaws 1012. Moving the jaws 1012 from the open position to the closed position allows a user to clamp down on and compress a tissue, such as a vessel. The handpiece 1001 can also allow a user to rotate the end effector 1002. For example, rotating the rotational actuator 1030 causes the end effector 1002 to rotate by rotating both the drive shaft 1026 and the outer shaft 1028 together.

With the tissue compressed between the jaws 1012, a user can depress the activation button 1036, such as to cause an electromagnetic or acoustic energy to be delivered to the end effector 1002, such as to an electrode or other transducer. Applying electromagnetic energy can help seal or otherwise affect the tissue being clamped. The electromagnetic energy can cause tissue to be coagulated, cauterized, sealed, ablated, desiccated, or can cause controlled necrosis. Examples of electrodes are described herein, but electromagnetic energy can be applied to any suitable electrode.

FIG. 1C illustrates a partial cross-sectional view of the forceps 1000 in a force limiting state. Shown here, the forceps 1000 can include a variable motion transfer assembly that can include the drive body 1052, the compressible member 1054, and the clip 1056. Together, the drive body 1052, the compressible member 1054, and the clip 1056, can move the drive shaft 1026 in response to the lever 1024 providing an input to a linkage between the lever 1024 and the drive body 1052. This can in turn provide force to the jaw 1012. The variable motion transfer assembly can allow for tailoring of the force provided by the forceps 1000 end effector 1002 on tissue or a vessel.

The components of the forceps 1000 shown in FIG. 1C can include the housing 1014, the lever 1024, the drive shaft 1026, the trigger 1034, the coupling link 1042, the drive link 1046, the drive body 1052, the compressible member 1054, the clip 1056, the outer hub 1060, a spool 1064, the cross pin 1066, and the trigger return spring 1068. Here, the drive body 1052 can include the body portion 1072, the anchor portion 1074 (including end-user-positionable seat 1076), and the window portion 1082. The spool 1064 can include a trigger return spring seat 1101. The spool 1064 is shown as one example of a motion transfer body to transmit motion received from an actuator to a shaft (e.g., received from trigger 1034 and transmitted to blade shaft 1032). The motion transfer body need not be spool-shaped, such as where the spool 1064 does not need to be rotatable.

Placing the compressible member 1054 in an over-travel position can allow for altering the force applied to the end effector 1002 jaws 1012. The variable motion transfer assembly can allow for a surgeon (or other operator) to adjust how much compression is on the spring 1054, and consequently how much force is applied through the jaws 1012. This user-adjustable compression force on the spring 1054 can control the maximum clamp force in the jaws of the end effector 1002.

In other words, the end-user-positionable seat 1076 drives the compressible member 1054, which drives the clip 1056, along with the drive body 1052. When the drive force supplied by the drive link 1046 is less than the preload force in the compressible member 1054, the compressible member 1054 acts like a rigid body and the ends of the compressible member 1054 move together. As such, the drive body 1052 moves proximally with respect to the housing 1014 and the clip 1056 moves proximally with respect to the housing 1014. Because the clip 1056 is longitudinally locked to the drive shaft 1026 at the first vertical slot 1070A and the second vertical slot 1070B, the drive shaft 1026 also moves proximally with respect to the housing 1014. As the drive shaft 1026 moves proximally (e.g., is retracted), the end effector 1002 becomes actuated. Here, actuating the end effector 1002 includes the jaws 1012 beginning to close. Thus, the force exerted by the end effector 1002 is controlled by the drive force, and can be used to limit the force of the jaws 1012 accordingly, such as to prevent damage to tissue therebetween.

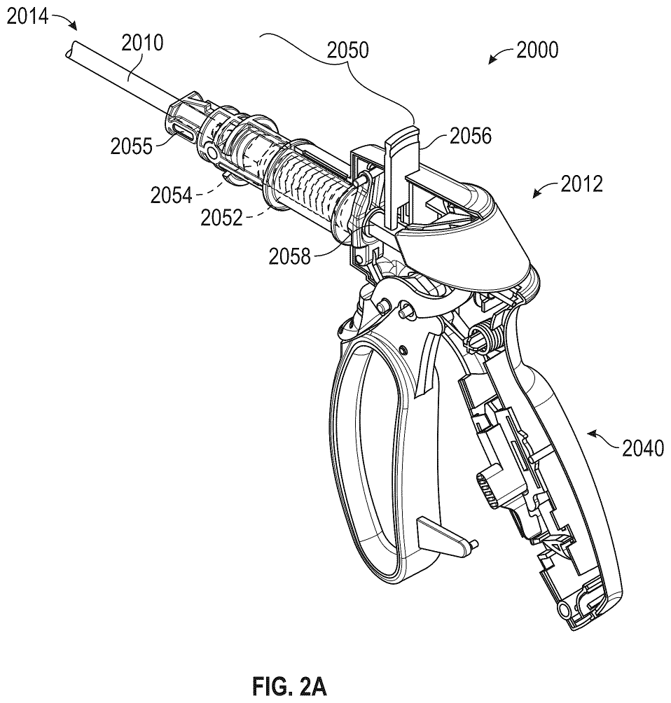

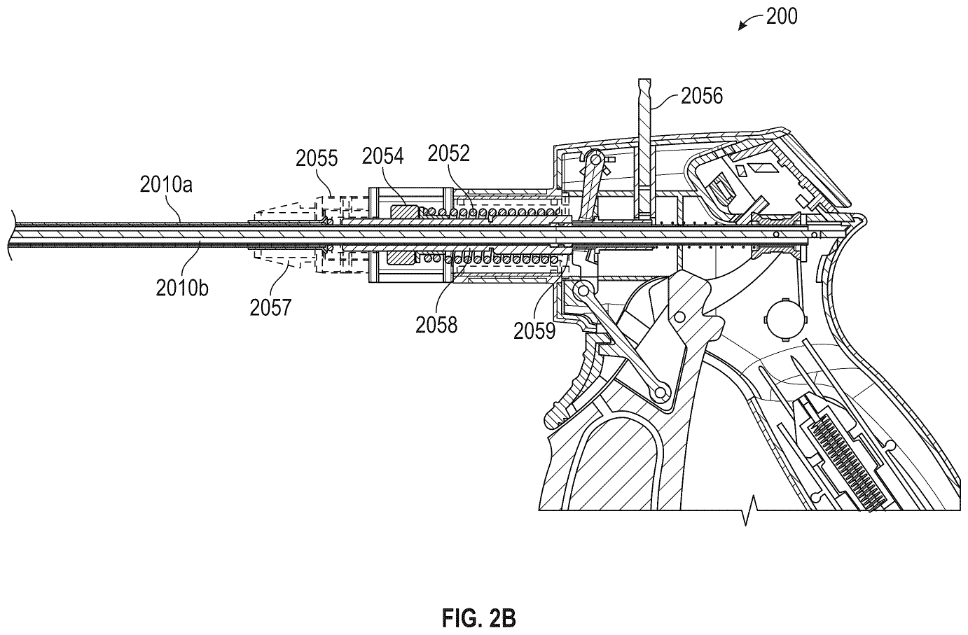

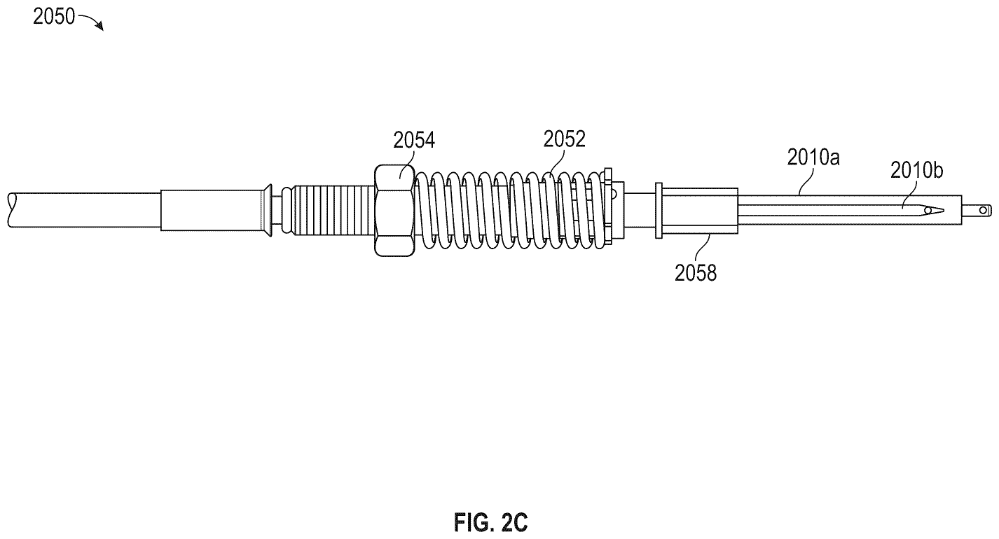

FIGS. 2A-2C illustrate views of a device 2000 having a variable motion transfer assembly 2050. FIG. 2A depicts a perspective view of the device 2000, while FIG. 2B shows a cross-sectional view of the device 2000. FIG. 2C depicts a close up view of the variable motion transfer assembly 2050. The device shown and discussed with reference to FIGS. 2A to 2C can allow for a surgeon (or other operator) alter the maximum allowed force exerted through the forceps.

The device 2000 can include a longitudinal shaft 2010 with a proximal portion 2012 and a distal portion 2014, having an end effector 2020 attached to and extending from the distal portion 2014. The device 2000 can further include a handpiece 2040 with a trigger 2042, operably connected to the end effector 2020, the handpiece attached to and extending from the proximal portion 2012.

The device 2000 can include a variable motion transfer assembly 2050 that can include a compressible member 2052, an end-user-positionable seat 2054, a hub 2055 with a knob 2057, a lock 2056, an adjustable slider 2058, and a clip 2059. The compressible member 2052 can be aligned with the longitudinal shaft 2010 and configured for applying a variable maximum bias force for communication to the end effector 2020. The end-user-positionable seat 2054 can be located against a first end of the compressible member 2052, the seat 2054 can be actuatable by an end user for varying the variable maximum bias force on the compressible member 2052. The end-user-positionable seat 2054 can be securable via the lock 2056, the adjustable slider 2058, and the clip 2059. The hub 2055 can at least partially cover or encapsulate the variable motion transfer assembly 2050, including the compressible member 2052 and the end-user-positionable seat 2054.

The longitudinal shaft 2010 can extend along the length of the device 2000 from the handpiece 2040 to the end effector 2020. The longitudinal shaft 2010 can host the variable motion transfer assembly 2050 on the proximal portion 2012 near the handpiece 2040. The longitudinal shaft 2010 can include an outer tube 2010a and an inner tube 2010b. The inner tube 2010b can be rotatable within the outer tube 2010a to allow for movement of the device 2000 during surgery. The handpiece 2040 can be operably connected to the end effector 2020 through the length of the longitudinal shaft 2010, such that the trigger 2042 can be used to affect the end effector 2020.

The variable motion transfer assembly 2050, similar to the motion transfer assembly including the drive shaft discussed above, can be used to actuate force to the end effector 2020 forceps 2022, by providing force down the length of the longitudinal shaft 2010 to the forceps 2022 themselves, such as described above with reference to FIGS. 1A to 1C. With the variable motion transfer assembly 2050, the amount of force exerted by the end effector 2020 can be varied, such as through altering the variable motion transfer assembly 2050.

The variable motion transfer assembly 2050 can include the compressible member 2052, the end-user-positionable seat 2054, the lock 2056, the adjustable slider 2058, and the clip 2059, and can be at least partially inside the hub 2055. The variable motion transfer assembly 2050 can be situated along the longitudinal shaft 2010. The compressible member 2052, the end-user-positionable seat 2054, the hub 2055, and the adjustable slider 2058 can be on or around the longitudinal shaft 2010. For example, the variable motion transfer assembly 2050 components can be on the longitudinal shaft 2010 just distal the handpiece 2040 to allow for interaction of the handpiece 2040 with the variable motion transfer assembly 2050.

In the device 2000, the compressible member 2052 can be aligned with the longitudinal shaft 2010 and configured for applying a variable maximum bias force for communication to the end effector 2020. The compressible member 2052 can be compressed or relaxed using the other components of the variable motion transfer assembly 2050, such as the end-user-positionable seat 2054. In the example of FIGS. 2A to 2C, the compressible member 2052 is a spring that can be biased through movement of the end-user-positionable seat 2054. In some cases, the compressible member 2052 can include a spring, a coiled spring, a leaf spring, a disk spring, a helical spring, an overtravel spring, a double spring, a pneumatic element, or any combination thereof.

The end-user-positionable seat 2054 can be located against a first end of the compressible member 2052. The seat 2054 can be actuatable by an end user for varying the variable maximum bias force. For example, the end-user-positionable seat 2054 can be physically moved distally or proximally along the longitudinal shaft 2010 to apply compression to the compressible member 2052, and subsequently provide additional or less force to the end effector 2020. Here, the end-user-positionable seat 2054 can be actuatable for providing a continuous change to the bias force.

In the example of FIGS. 2A to 2C, the end-user-positionable seat 2054 can include a hex nut within the hub 2055 and situated on the adjustable slider 2058. The hub 2055 can include an over mold at least partially encapsulating the variable motion transfer assembly 2050. In some cases, the end-user-positionable 2054 seat can include a rotational element, such as a nut, a hexagonal nut, a washer, a wedge, or a cam.

In FIGS. 2A to 2C, the end-user-positionable seat 2054 hex nut can work with the adjustable slider 2058. For example, as shown in FIG. 2C, the adjustable slider 2058 can be threaded to allow the hex nut to increase or decrease the preload force on the compressible member 2052 spring. As such, the adjustable slider 2058 can be adjustable to help release or secure the compressible member 2052. The adjustable slider 2058 itself can be held in place, for example, by a lock 2056. The lock 2056 can include, for example, a holder, a clip, or a slider holder as shown in FIGS. 2A-2B.

The end-user-positionable seat 2054 hex nut can abut a first side of the compressible member 2052 spring. The opposing end of the compressible member 2052 spring can be secured, for example, by the proximal end of the hub 2055. The hub 2055 can be stationary, such that movement of the adjustable slider 2058 hex nut compresses or decompresses the compressible member 2052 spring therebetween.

Shown in the cross-section of FIG. 2B, the clip 2059 can help secure the adjustable slider 2058 to the inner tube 2010b of the longitudinal shaft 2010 along a longitudinal axis. The hub 2055 can include the knob 2057 secured to the outer tube 2010a. This can permit the end-user-positionable seat 2054 hex nut to rotate while the lock 2056 holds the adjustable slider 2058 in place. In use, when the end-user-positionable seat 2054 hex nut is moved laterally along the threads on the adjustable slider 2058, the compressible member 2052 spring preload increases and decreases, effectively varying the jaw force in the end effector 2020.

For the device shown and discussed with reference to FIGS. 2A to 2C, adjusting the preload onto the compressible member 2052 spring effectively varying the jaw force when the clamp lever is fully engaged. For example, a surgeon can lower the lock 2056 to lock the adjustable slider 2058 in place such that the adjustable slider 2058 will not rotate. Then, the surgeon can rotate the knob 2057, which in turn would rotate the end-user-positionable seat 2054 hex nut inside the hub 2055. This can increase or decrease the compressible member 2052 spring preload. Once the device jaw force has been set, such as after a desired number of rotations, the surgeon can lock or completely remove the lock 2056. In this case, the longitudinal shaft 2010 is now free to rotate and the device 2000 is ready for use in surgery with the desired jaw force.

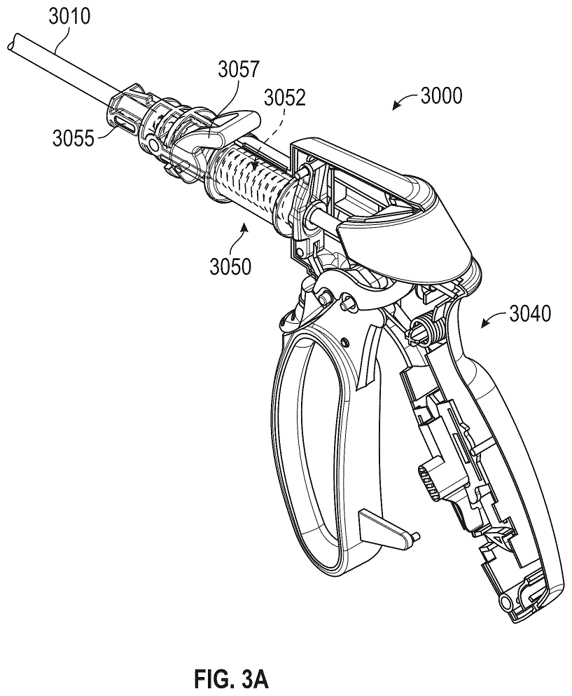

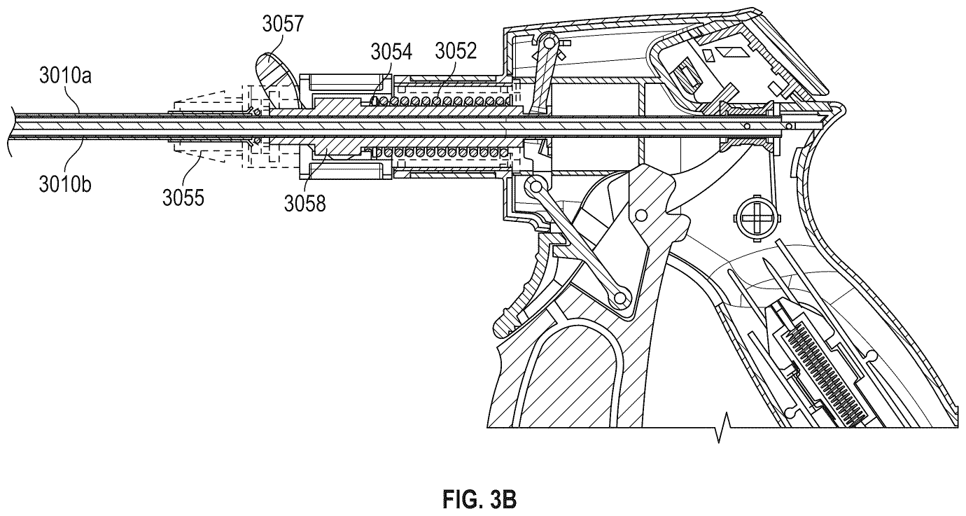

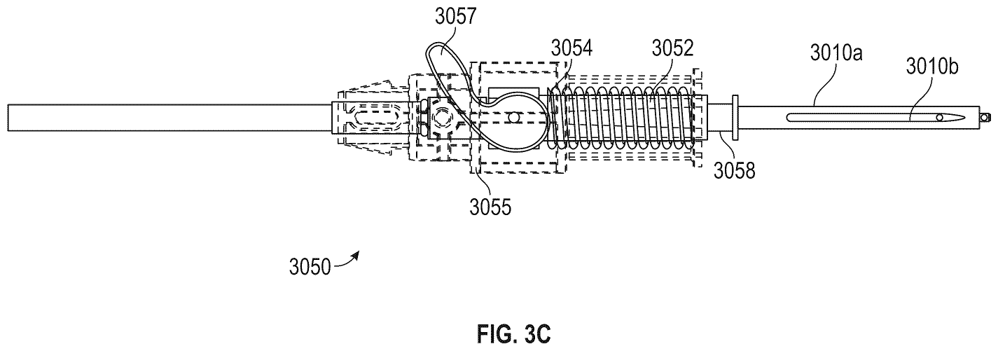

FIGS. 3A-3C illustrate views of a device 3000 having a variable motion transfer assembly. The device 3000 is similar to the device 2000 discussed above, however, the configuration of device 3000 allows for a stepped change in the desired jaw force. In the device 3000, the preload on the spring can be adjusted discretely in two or more specific positions.

FIG. 3A depicts a perspective view of the device 3000, while FIG. 3B shows a cross-sectional view of the device 3000. FIG. 3C depicts a close up view of the variable motion transfer assembly 3050.

The device 3000 can include a longitudinal shaft 3010 with a proximal portion 3012 and a distal portion 3014, having an end effector 3020 attached to and extending from the distal portion 3014. The device 3000 can further include a handpiece 3040 with a trigger 3042, operably connected to the end effector 3020, the handpiece attached to and extending from the proximal portion 3012.

The device 3000 can include a variable motion transfer assembly 3050 that can include a compressible member 3052, an end-user-positionable seat 3054, a hub 3055 with a cam lever 3057, and an adjustable slider 3058. The compressible member 3052 can be aligned with the longitudinal shaft 3010 and can be configured for applying a variable maximum bias force for communication to the end effector 3020. The end-user-positionable seat 3054 can be located against a first end of the compressible member 3052. The seat 3054 can be actuatable by an end user for varying the bias force on the compressible member 3052. The end-user-positionable seat 3054 can be securable via the cam lever 3057. The hub 3055 can at least partially cover or encapsulate the variable motion transfer assembly 3050, including the compressible member 3052 and the end-user-positionable seat 3054.

The longitudinal shaft 3010 can extend along the length of the device 3000 from the handpiece 3040 to the end effector 3020. The longitudinal shaft 3010 can host the variable motion transfer assembly 3050 on the proximal portion 3012 near the handpiece 3040. The longitudinal shaft 3010 can include an outer tube 3010a and an inner tube 3010b. The inner tube 3010b can be rotatable within the outer tube 3010a to allow for movement of the device 3000 during surgery. The handpiece 3040 can be operably connected to the end effector 3020 through the length of the longitudinal shaft 3010, such that the trigger 3042 can be used to affect the end effector 3020.

The variable motion transfer assembly 3050, similar to the motion transfer assembly including the drive shaft discussed above, can be used to actuate force to the end effector 3020 forceps 3022, by providing force down the length of the longitudinal shaft 3010 to the forceps 3022 themselves, such as described above with reference to FIGS. 1A to 1C. With the variable motion transfer assembly 3050, the amount of force exerted by the end effector 3020 can be varied, such as through altering the variable motion transfer assembly 3050.

The variable motion transfer assembly 3050 can include the compressible member 3052, the end-user-positionable seat 3054, the cam lever 3057, and the adjustable slider 3058, and is at least partially enclosed by the hub 3055.

FIG. 3B depicts a cross-sectional view of the device 3000 and the variable motion transfer assembly 3050. The variable motion transfer assembly 3050 can include a cam-operated or other power seal device. When the cam lever 3057 is rotated counter clockwise, the cam surface pushes on the end-user-positionable seat 3054, which here can include a washer. The cam configuration can be used to move the washer between two different positions, and can consequently compress or decompress the compressible member 3052, such as a an overtravel spring. In the first position, where the cam surface pushes on the end-user-positionable seat 3054, the compressible member 3052 is compressed. This position creates a high jaw forces when compared to a cam lever 3057 position shown in FIG. 3A.

FIG. 3C shows a closer view of the variable motion transfer assembly 3050, and specifically the power seal shaft 3010 assembly. Here, the cam has been moved forward with the cam lever 3057 into the higher jaw force position. The compressible member 3052 is further compressed, thereby increasing the jaw force.

In use, the device 3000 variable motion transfer assembly 3050 can allow for moving between a standard (e.g., default) jaw force mode and a higher jaw force mode. In the standard jaw force mode, the cam lever 3057 is in the “back” position, such as to relieve compression on the compressible member 3052. For example, the surgeon can use the device 3000, at the default setting, and may come across a vessel that requires higher jaw force to address. In this case, the surgeon can move the cam lever 3057 to the forward position, such as to switch the device 3000 variable motion transfer assembly 3050 into the higher jaw force mode by compressing the compressible member 3052. In this example, the longitudinal shaft 3010 can rotate in either the default or the higher haw force mode. The device 3000 can then be used in either the default or higher jaw force mode.

In both the examples of device 2000 and device 3000, the surgeon (or other operator) of the device can be notified of the maximum amount of jaw force in effect. Similarly, a generator, such as a system or device connected to the device to provide electromagnetic energy thereto, can be notified of whether the jaw force being used is in a standard/default mode, or a higher jaw force mode. In some cases, a controller or user interface can receive, provide, or use information regarding the maximum amount of jaw force in effect. This can be indicated to the surgeon via one or more of labels, scales, colored windows, or other visual indicators, such as physically showing the location and tension of the compressible member.

One or more sensors (e.g., sensor 2090) can be used as an input to a generator program to inform the generator about the jaw force mode that the device is in, such as one or more discrete modes, or a continuous mode. This can allow for the generator to be informed about the jaw force mode. In response, the generator can tailor energy output to the jaws. The sensor can include any suitable sensors, including but not limited to mechanical or electrical sensors. In some examples, a mechanical or electrical contact, such as a tactile switch, dome switch, a sensor, or other, can provide an input to a generator program to indicate a jaw force mode.

Other types of sensors can include proximity sensors, optical (e.g. fiber optic), or electromagnetic sensors, however any suitable sensor to provide jaw force mode information can be used. Alternatively, the user could manually input into a user interface, the jaw force mode of the device. This can be received by a controller and/or generator connected to the device. In this case, any electromagnetic energy provided to the forceps, such as a waveform provided by the generator to the device, can be optimized against (e.g., based on) the amount of force exerted on the tissue during surgery. In an example, the forceps may include an electrode or cutting blade to which such electromagnetic energy is applied. The generator can use the jaw force mode to determine what electromagnetic energy to transmit. The generator can also use the jaw force information to determine any type of energy to send to the jaw, such as any one or more of: electromagnetic energy, radiofrequency, microwave energy, ultrasonic energy, thermal energy, light energy, laser energy.

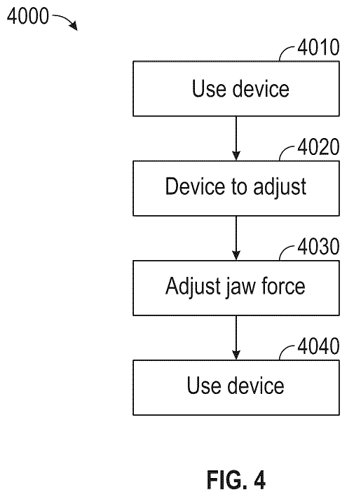

FIG. 4 illustrates a method 4000 of using a forceps having a variable motion transfer assembly.

At block 4010, the surgeon can use the device with a variable motion transfer assembly, such as device 2000 or device 3000 discussed above. The surgeon can, for example, begin a procedure, such as a laparoscopic procedure, with the device in a default mode. In such a default mode, the device can provide a specified maximum jaw force.

At block 4020, the surgeon may come across a vessel that is of differing size or complexity than other vessels being addressed during the procedure using the device forceps. For example, the device can initially be used to cut, coagulate, or seal vessels with a diameter of less than about 7 mm. However, if a vessel with a larger diameter is encountered, more jaw force may be desired. In some cases, other or additional factors, such as hemostasis or tissue type, can factor into the amount of jaw force desired.

In this case, at block 4030, the surgeon can adjust the jaw force of the device. For example, this can be done by moving a user-positionable seat against a compressible member, such as those discussed with reference to FIGS. 2A to 2C above. Such a configuration may be used to establish a continuously changeable force that the surgeon can then select and thereby lock in place for full use of the device. In some cases, this can be accomplished in a stepped fashion, such as by using a cam configuration, like that discussed with reference to FIGS. 3A to 3C above. In either case, the surgeon can adjust the jaw force to a new desired force. The surgeon can then continue the procedure at block 4040, without the need for additional or differing instruments or clips.

For various types of devices with alterable jaw force and various jaw force sensing methods, it is important that the surgeon and the generator know whether the jaw force is in the standard or high jaw force mode. This could be indicated to the surgeon via labels, scales, or colored windows showing the relative position of the overtravel spring.

Preferably, the generator would also be aware of the jaw force mode. Mechanical or electrical contacts, such as a tactile switch, dome switch, or other means would be used as an input to the generator program to indicate jaw force mode. The generator can use the jaw force information to modify any aspect of the generator performance. For example, the generator waveform would then be modified (e.g., optimized) based on the amount of force exerted on the tissue during surgery. Modifying the generator waveform can include optimizing the radiofrequency therapeutic output to match the jaw force mode. Any other aspect of the generator waveform or other operating and performance characteristics of the generator can also be modified based on the jaw force information, to more accurately assess the tissue being treated, adjust generator settings, and to improve treatment, and is not limited to modifying only the waveform, or modifying based on discrete jaw force modes.

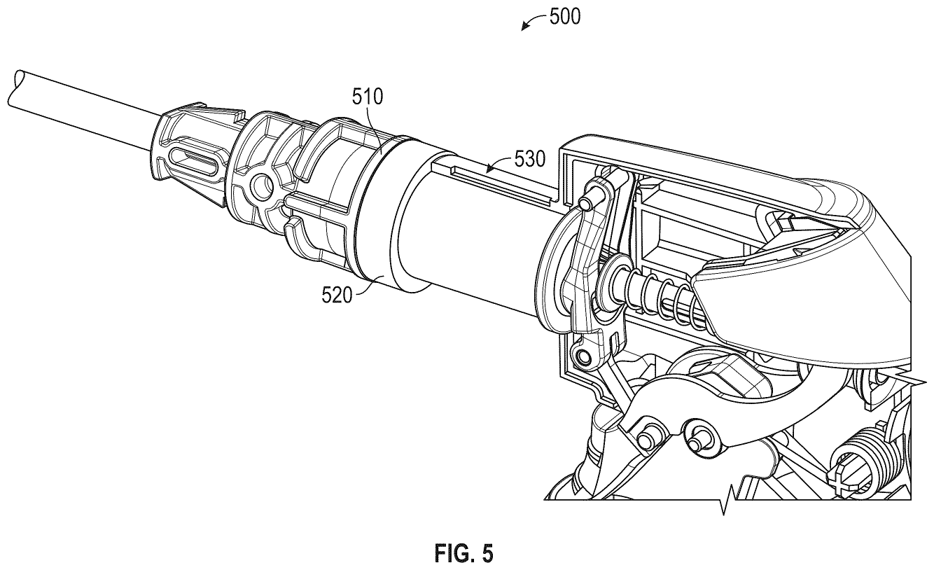

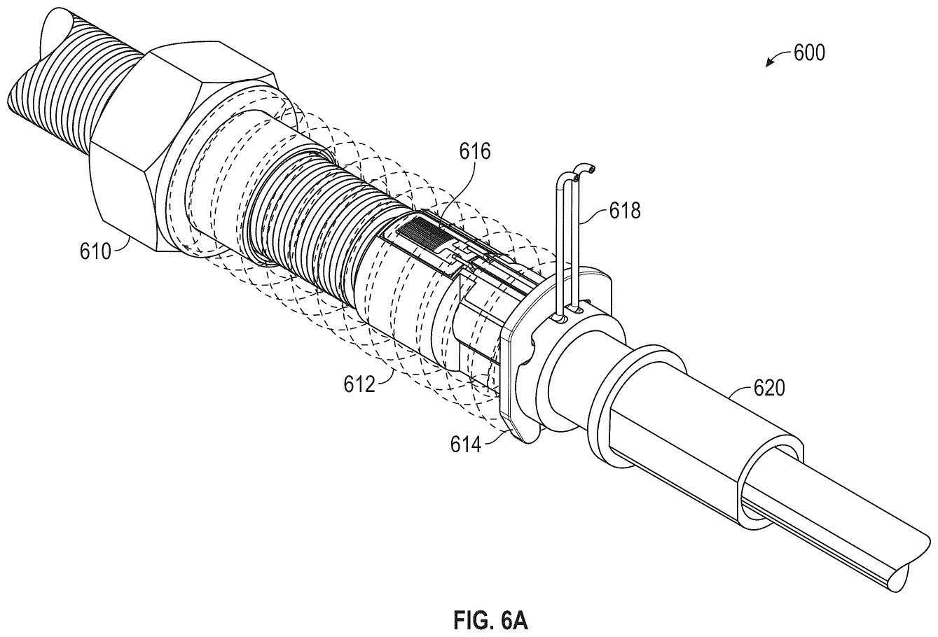

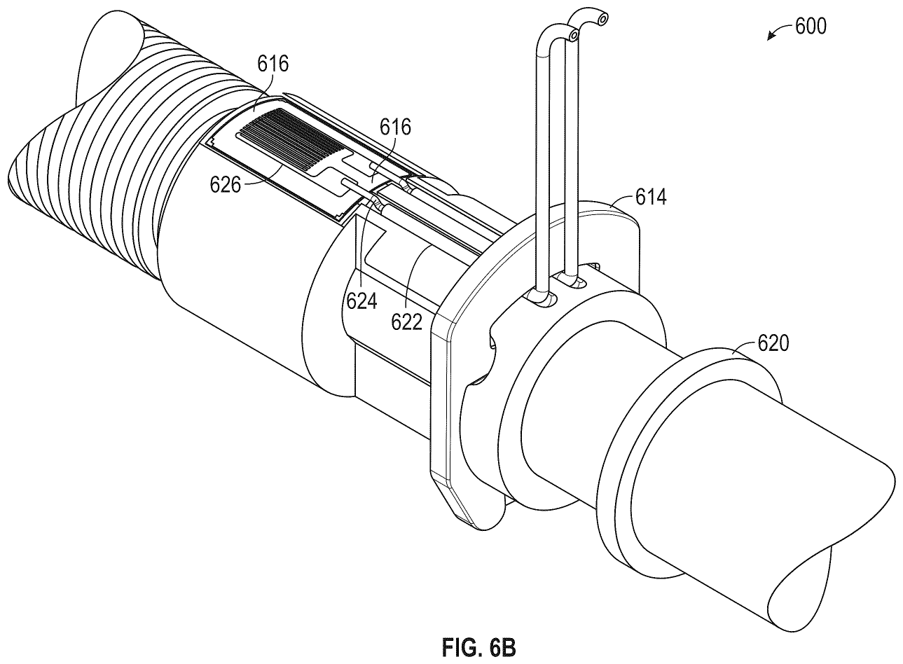

FIGS. 5 to 9E below depict devices with sensor designs for measuring jaw force settings. For example, FIG. 5 depicts an example with a load cell, while FIGS. 6A to 6B depict an example with a strain gauge. Discussed here are various examples of ways to determine the jaw force mode in such a device. Ways to determine the jaw force mode can include measuring a pre-load force on the spring that controls the maximum jaw force using a sensor such as, but not limited to, a load cell or a strain gauge. The load cell or strain gauge can be used to measure the pre-load force on the overtravel spring. A benefit of the load cell or strain gauge is having the ability to determine the actual force on the spring. This being useful in the case of a device that has continuously adjustable jaw force, but also still applicable to discrete adjustable jaw force devices as well.

FIG. 5 depicts an example jaw 500 with a load cell 520. The jaw 500 can also include a hub 510 and housing 530. FIG. 5 depicts an example where the pre-load force on the spring can be measured using a load cell. The load cell can be placed in various locations to measure the preload on the spring. One example location for load cell placement can be between the hub 510 and the housing 530.

When the sensor measures the preload on the spring being within a first force range, that can represent a first jaw force mode, and a second force range can represent a second jaw force mode. First and second modes are provided for illustrative purposes, but any number of modes can be provided. Increasing the number of modes can be particularly advantageous in the case where there are more than two modes or in the case of a continuously adjustable jaw force embodiment described herein.

FIGS. 6A-6B depict an example device 600 with a strain gauge. The device 600 can include a hex nut 610, an overtravel spring 612, a clip 614, a linear strain gauge 616, strain gauge wires 618, an adjustable slider 620, a trough 622 for the wires 618, an S bend 624 for use with decouple strain gauge from wires, and a carrier 626 bonded to the adjustable slider 620 using epoxy.

The example of device 600 is a lower cost way to sense the pre-load on the spring 612 to determine the jaw force mode (e.g., maximum jaw force setting) with one or more strain gauge 616. FIG. 7 depicts an additional example of such a strain gauge 700. One or more strain gauge 616 can be placed in various locations to determine the maximum jaw force setting. In some examples, a strain gauge 616 can include a Wheatstone bridge circuit.

In the example device 600, the linear strain gauge 616 is mounted directly onto the adjustable slider 620. The carrier is bonded to the underlying material using epoxy. In this embodiment, the strain gauge 616 carrier is recessed on the adjustable slider and lies inside the overtravel spring's 612 inside diameter.

The strain gauge's 616 principal axis has been aligned axially to the adjustable slider 620 to directly measure the compressive force pre-load. The strain gauge 616 is decoupled from the wires 618 connecting to the gauge itself. Here, there is slight “S” bend 624 near the solder tabs of the strain gauge as the wires go into the slider troughs. This helps minimize movement in the wires from effecting the strain gauge measurement. The two wires can continue down the troughs 622, go under the clip 614 and emerge upward so as not to interfere with the 4-bar clamp lever mechanism inside the device handle. A zoomed-in view of this is shown in FIG. 6B.

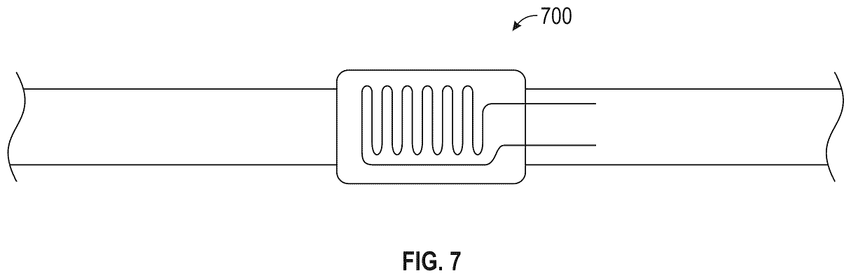

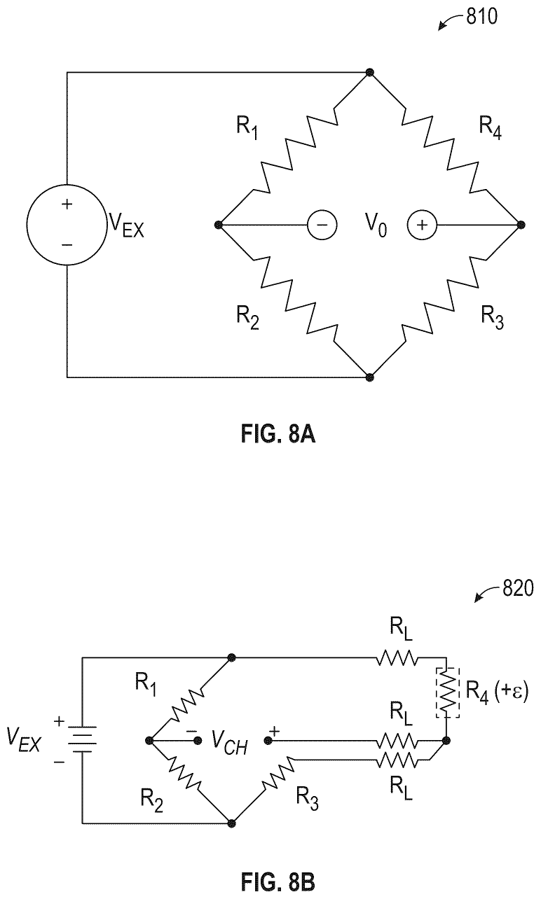

The strain gauge 616 can be used in a quarter-bridge Wheatstone bridge circuit, such as those shown in FIG. 8A-8B. This is a parallel voltage-divider circuit that includes of four resistors and a DC excitation voltage (Vex). The 4 resistors can be strain gauges or a combination of resistors and strain gauges. When the circuit is “balanced”, the voltage measured at V0 is zero. In the quarter-bridge configuration, resistor R4 would be the linear strain gauge, here we have chosen a 350 ohm gauge resistance. A change in the compressive force on the adjustable slider, due to a change in the spring compressive spring pre-load, would unbalance the bridge, resulting in a change to V0 proportional to strain. This small voltage signal (e.g. 2 mV/V) is typically amplified and conditioned such that it can be used by standard analog-to-digital circuitry. The amplification and conditioning would likely be done by the generator circuitry to minimize the cost of the disposable devices.

It shall be noted that although only two wires are shown in FIGS. 6A-6B, it is known in electronics that a third wire is often required to compensate for wire resistance leading to the strain gauge, particularly when the strain gauge is a long distance away from the other resistors that make-up the Wheatstone bridge (i.e. resistors R₁, R₂, and R₃). In an illustrative embodiment, one or more strain gauge or Wheatstone bridge can be located along a shaft of the device as shown below.

FIGS. 8A-8B depict examples of such Wheatstone bridges 810 and 820, e.g., a circuit where the resistors are strain gauges. FIG. 8B depicts an example of a quarter bridge Wheatstone circuit and third wire used to minimize the apparent resistance due to the long wire leads connecting to R₄. The equation relating unamplified V0 (VCH) to the strain, F, is shown below:

$$strain ( ε ) = \frac{- 4 {V}_{r}}{GF ( 1 + 2 {V}_{r} )} \times ( 1 + \frac{{R}_{L}}{Rg} )$$

Where RL is the lead wire resistance, Rg is the gauge resistance (i.e. 350 ohms). GF is the gauge factor and Vr is the voltage ratio to relate the V0 (VCH) to the excitation voltage as shown in the equation below:

$${V}_{r} = ( \frac{{V}_{CH} ( strained ) - {V}_{CH} ( unstrained )}{{V}_{EX}} )$$

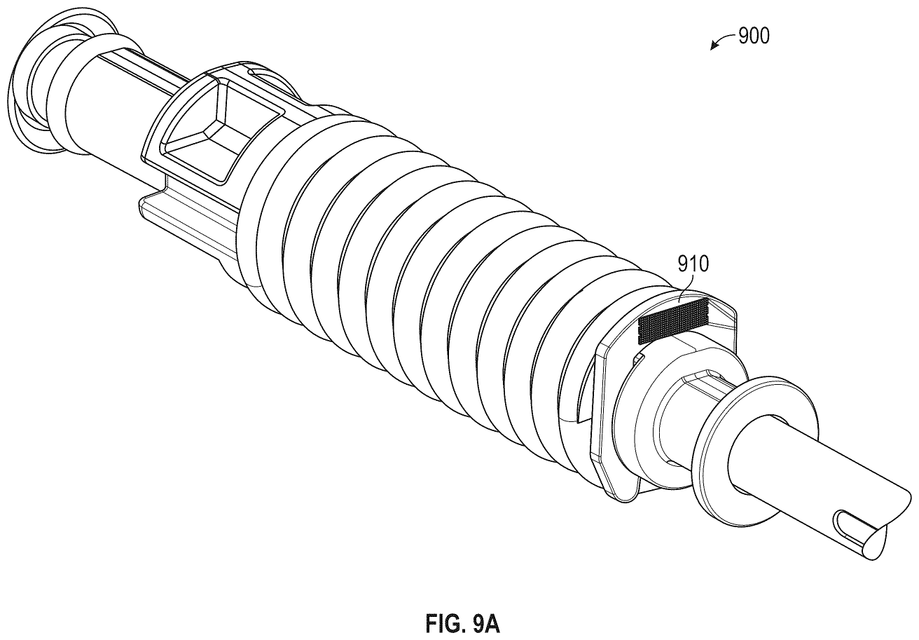

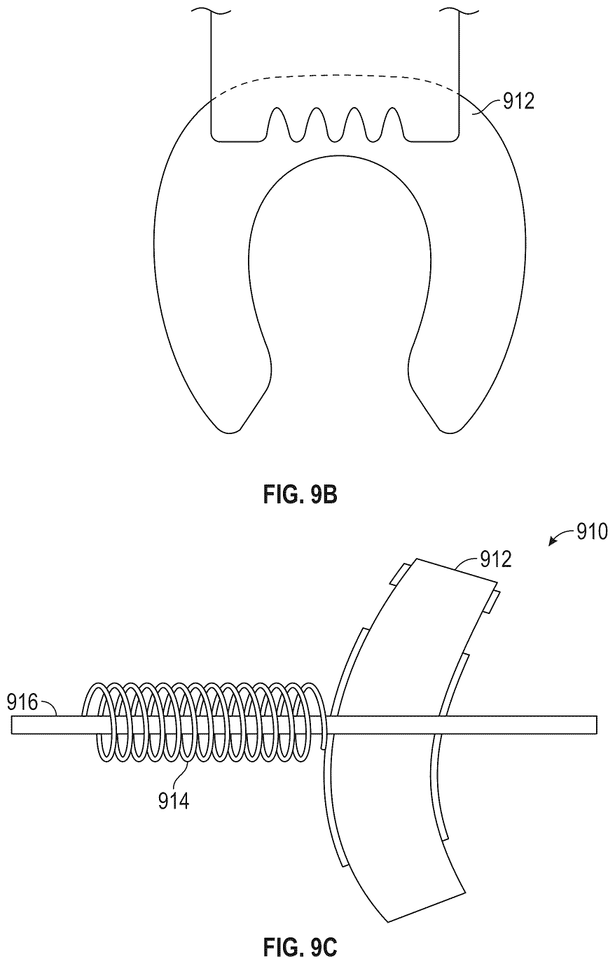

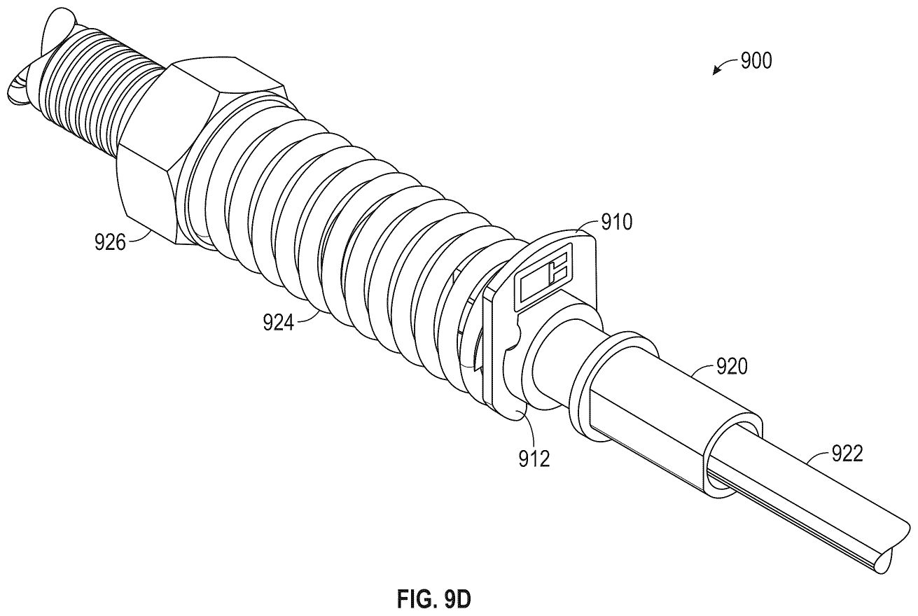

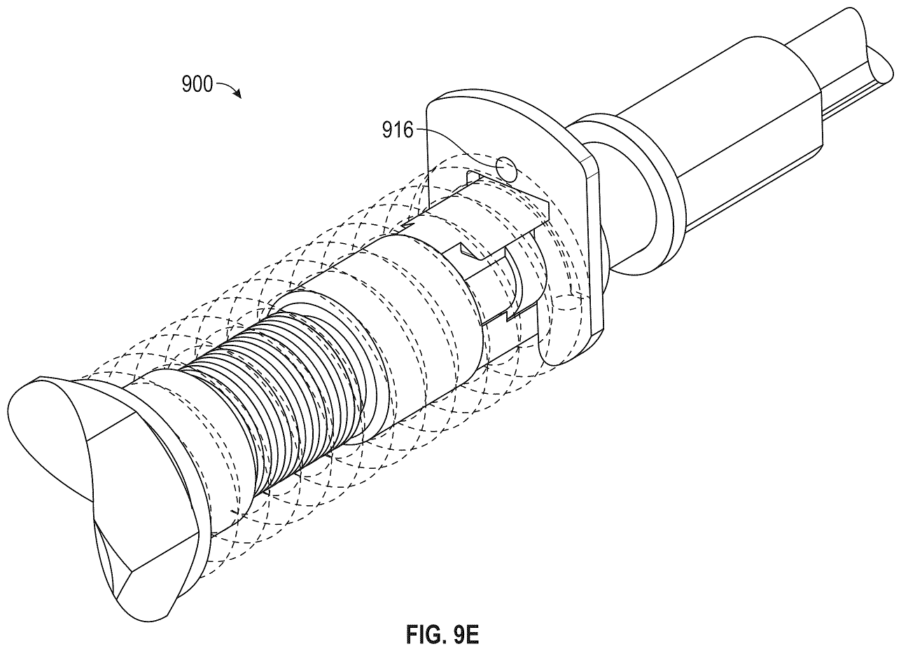

FIG. 9A-9E depict another example of a device 900 with a strain gauge 910, clip 912, spring 914, and shaft 916. Here, the strain gauge 910 can be located on the clip. The benefit of such as design is that no additional component need be added to the system. By adding a strain gauge 910 to the clip 912, other functional aspects provided by the clip 912 are maintained, but then the clip 912 can have the added benefit that it can also be used to sense the pre-load on the spring 914 in an indirect manner. Here, as the spring 914 pre-load varies by adjusting the nut (continuous) or moving the lever (discrete), the clip 912 will experience a varying bending force. This occurs because the clip has been specially designed to bend along two contact lines and a bump on the clip.

The clip 912 has a strain gauge mounted to its face as shown in the diagram. The opposite side of the clip has bump along its centerline. The bump provides a pivot point for the clip to bend equally on each side along the vertical plane (running through the center axis of the adjustable slider) as the spring 914 pushes on the left and right sides of the clip 912. The folding motion of the clipped (when viewed from the top) creates a bending strain that is proportional to the force of the spring 914 contacting it. The strain gauge could be use in either a quarter bridge circuit as described earlier.

Alternatively, other sensor types for monitoring of force can include an LVDT sensor (e.g., a linear variable differential transformer), which as position changes, the resistance changes, and the sensor sees a voltage change. Other sensor types could include encoder, optical encoder, magnetic encoder, optical switch, proximity detector sensors, or combinations thereof.

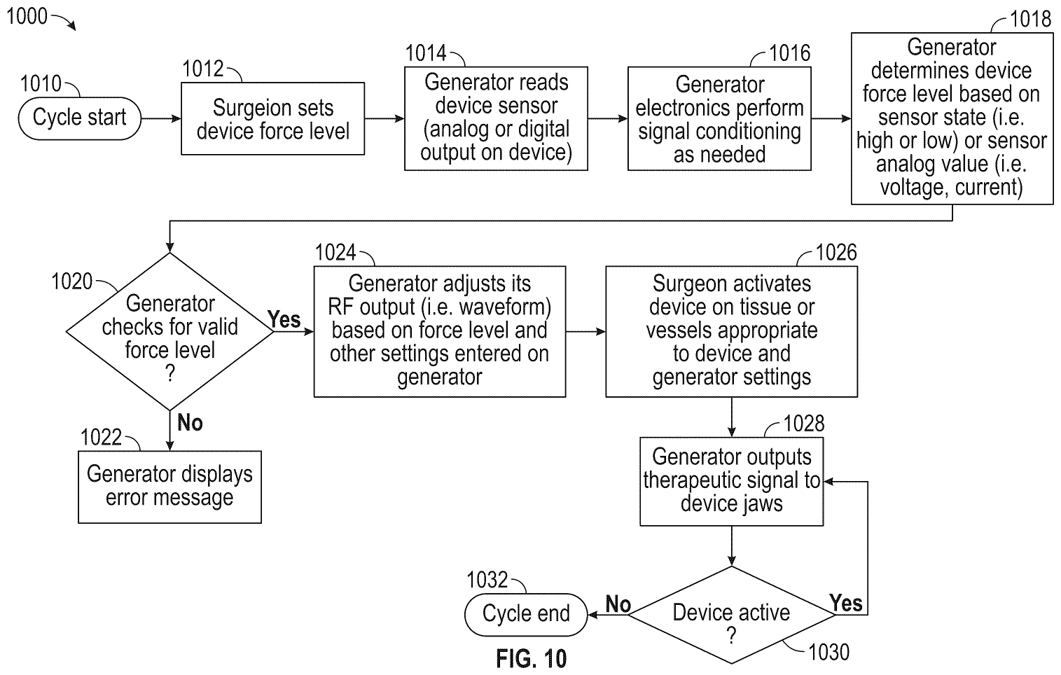

FIG. 10 depicts a method 1001 of sensing and communicating jaw force level or state. In this method, through blocks 1011 to 1033, the generator or device can detect the jaw force level. Here, the surgeon can adjust the jaw force setting, the sensor in the device can send a signal to the generator about those force settings, the sensor signal can be conditioned, the generator can adjust the appropriate waveform, and the generator can send a therapeutic signal to the device jaws.

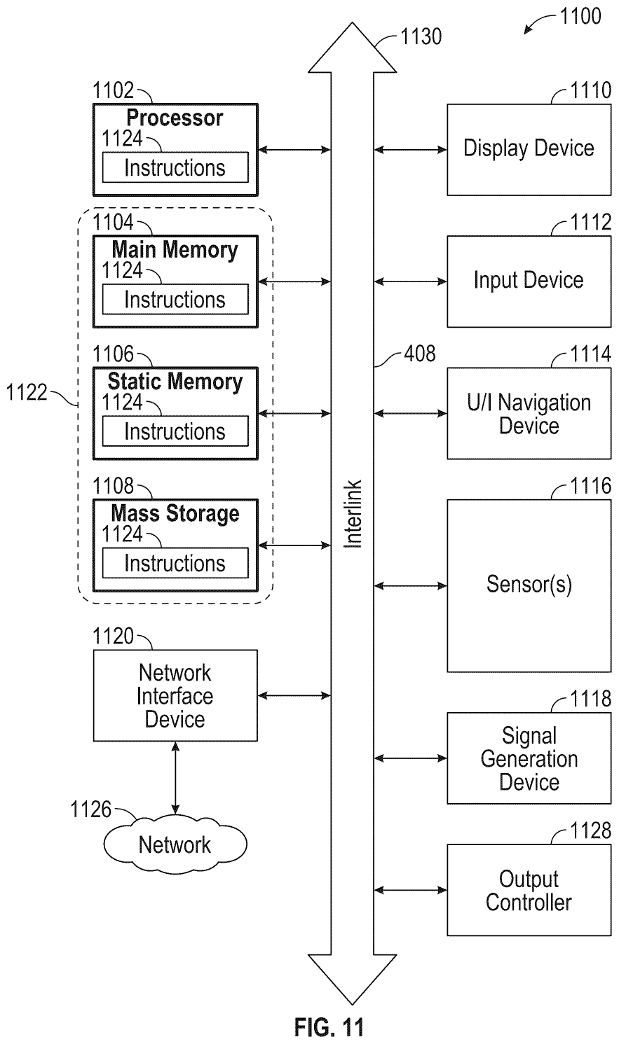

FIG. 11 illustrates a block diagram of an example machine 1100 upon which any one or more of the techniques (e.g., methodologies) discussed herein may perform. Examples, as described herein, may include, or may operate by, logic or a number of components, or mechanisms in the machine 1100. Circuitry (e.g., processing circuitry) is a collection of circuits implemented in tangible entities of the machine 1100 that include hardware (e.g., simple circuits, gates, logic, etc.). Circuitry membership may be flexible over time. Circuitries include members that may, alone or in combination, perform specified operations when operating. In an example, hardware of the circuitry may be immutably designed to carry out a specific operation (e.g., hardwired). In an example, the hardware of the circuitry may include variably connected physical components (e.g., execution units, transistors, simple circuits, etc.) including a machine readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuitry in hardware via the variable connections to carry out portions of the specific operation when in operation. Accordingly, in an example, the machine readable medium elements are part of the circuitry or are communicatively coupled to the other components of the circuitry when the device is operating. In an example, any of the physical components may be used in more than one member of more than one circuitry. For example, under operation, execution units may be used in a first circuit of a first circuitry at one point in time and reused by a second circuit in the first circuitry, or by a third circuit in a second circuitry at a different time. Additional examples of these components with respect to the machine 1100 follow.

In alternative embodiments, the machine 1100 may operate as a standalone device or may be connected (e.g., networked) to other machines. In a networked deployment, the machine 1100 may operate in the capacity of a server machine, a client machine, or both in server-client network environments. In an example, the machine 1100 may act as a peer machine in peer-to-peer (P2P) (or other distributed) network environment. The machine 1100 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

The machine (e.g., computer system) 1100 may include a hardware processor 1102 (e.g., a central processing unit (CPU), a graphics processing unit (GPU), a hardware processor core, or any combination thereof), a main memory 1104, a static memory (e.g., memory or storage for firmware, microcode, a basic-input-output (BIOS), unified extensible firmware interface (UEFI), etc.) 1106, and mass storage 1108 (e.g., hard drives, tape drives, flash storage, or other block devices) some or all of which may communicate with each other via an interlink (e.g., bus) 1130. The machine 1100 may further include a display unit 1110, an alphanumeric input device 1112 (e.g., a keyboard), and a user interface (UI) navigation device 1114 (e.g., a mouse). In an example, the display unit 1110, input device 1112 and UI navigation device 1114 may be a touch screen display. The machine 1100 may additionally include a storage device (e.g., drive unit) 1108, a signal generation device 1118 (e.g., a speaker), a network interface device 1120, and one or more sensors 1116, such as a global positioning system (GPS) sensor, compass, accelerometer, or other sensor. The machine 1100 may include an output controller 1128, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

Registers of the processor 1102, the main memory 1104, the static memory 1106, or the mass storage 1108 may be, or include, a machine readable medium 1122 on which is stored one or more sets of data structures or instructions 1124 (e.g., software) embodying or utilized by any one or more of the techniques or functions described herein. The instructions 1124 may also reside, completely or at least partially, within any of registers of the processor 1102, the main memory 1104, the static memory 1106, or the mass storage 1108 during execution thereof by the machine 1100. In an example, one or any combination of the hardware processor 1102, the main memory 1104, the static memory 1106, or the mass storage 1108 may constitute the machine readable media 1122. While the machine readable medium 1122 is illustrated as a single medium, the term “machine readable medium” may include a single medium or multiple media (e.g., a centralized or distributed database, and/or associated caches and servers) configured to store the one or more instructions 1124.

The term “machine readable medium” may include any medium that is capable of storing, encoding, or carrying instructions for execution by the machine 1100 and that cause the machine 1100 to perform any one or more of the techniques of the present disclosure, or that is capable of storing, encoding or carrying data structures used by or associated with such instructions. Non-limiting machine readable medium examples may include solid-state memories, optical media, magnetic media, and signals (e.g., radio frequency signals, other photon based signals, sound signals, etc.). In an example, a non-transitory machine readable medium comprises a machine readable medium with a plurality of particles having invariant (e.g., rest) mass, and thus are compositions of matter. Accordingly, non-transitory machine-readable media are machine readable media that do not include transitory propagating signals. Specific examples of non-transitory machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

In an example, information stored or otherwise provided on the machine readable medium 1122 may be representative of the instructions 1124, such as instructions 1124 themselves or a format from which the instructions 1124 may be derived. This format from which the instructions 1124 may be derived may include source code, encoded instructions (e.g., in compressed or encrypted form), packaged instructions (e.g., split into multiple packages), or the like. The information representative of the instructions 1124 in the machine readable medium 1122 may be processed by processing circuitry into the instructions to implement any of the operations discussed herein. For example, deriving the instructions 1124 from the information (e.g., processing by the processing circuitry) may include: compiling (e.g., from source code, object code, etc.), interpreting, loading, organizing (e.g., dynamically or statically linking), encoding, decoding, encrypting, unencrypting, packaging, unpackaging, or otherwise manipulating the information into the instructions 1124.

In an example, the derivation of the instructions 1124 may include assembly, compilation, or interpretation of the information (e.g., by the processing circuitry) to create the instructions 1124 from some intermediate or preprocessed format provided by the machine readable medium 1122. The information, when provided in multiple parts, may be combined, unpacked, and modified to create the instructions 1124. For example, the information may be in multiple compressed source code packages (or object code, or binary executable code, etc.) on one or several remote servers. The source code packages may be encrypted when in transit over a network and decrypted, uncompressed, assembled (e.g., linked) if necessary, and compiled or interpreted (e.g., into a library, stand-alone executable etc.) at a local machine, and executed by the local machine.

The instructions 1124 may be further transmitted or received over a communications network 1126 using a transmission medium via the network interface device 1120 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), LoRa/LoRaWAN, or satellite communication networks, mobile telephone networks (e.g., cellular networks such as those complying with 3G, 4G LTE/LTE-A, or 5G standards), Plain Old Telephone (POTS) networks, and wireless data networks (e.g., Institute of Electrical and Electronics Engineers (IEEE) 702.11 family of standards known as Wi-Fi®, IEEE 702.15.4 family of standards, peer-to-peer (P2P) networks, among others. In an example, the network interface device 1120 may include one or more physical jacks (e.g., Ethernet, coaxial, or phone jacks) or one or more antennas to connect to the communications network 1126. In an example, the network interface device 1120 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 1100, and includes digital or analog communications signals or other intangible medium to facilitate communication of such software. A transmission medium is a machine readable medium.

A numbered list of examples follows, with examples that refer to other examples incorporating by reference the recitations from such other examples being referred to.

Example 1 can include a medical device. The medical device can comprise a longitudinal shaft, having a proximal portion and a distal portion. An end effector can be attached to and can extend from the distal portion. A compressible member can be aligned with the longitudinal shaft. The compressible member can be configured for applying a variable maximum bias force for communication to the end effector. An end-user-positionable seat can be located against a first end of the compressible member. The seat can be actuatable by an end-user such as for varying the variable maximum bias force.

In Example 2, the subject matter of Example 1 can optionally include the compressible member comprising one or more of a spring, a coiled spring, a leaf spring, a disk spring, a helical spring, an overtravel spring, a double spring, a rubber “spring”, or a pneumatic element.

In Example 3, the subject matter of any one or more of Examples 1-2 can optionally include the end-user-positionable seat being actuatable for providing a continuous change to the bias force.

In Example 4, the subject matter of any one or more of Examples 1-3 can optionally include the end-user-positionable seat being actuatable for providing a stepped change to the bias force.

In Example 5, the subject matter of any one or more of Examples 1-4 can optionally include the end-user-positionable seat comprising a knob actuatable for rotating and moving the end-user positionable seat.

In Example 6, the subject matter of any one or more of Examples 1-5 can optionally include the end-user-positionable seat comprising one or more of a nut, a hexagonal nut, a washer, or a cam.

In Example 7, the subject matter of any one or more of Examples 1-6 can optionally include a hub at least partially surrounding the end-user-positionable seat and the compressible member.

In Example 8, the subject matter of any one or more of Examples 1-7 can optionally include the end-user-positionable seat comprising a slider aligning with at least a portion of the longitudinal shaft. The slider can be moveable to release or secure the compressible member. A lock can be included. The lock can be actuatable by an end-user. The lock can include end-user-selectable first and second states such as for respectively inhibiting or allowing the varying of the variable maximum bias force.

In Example 9, the subject matter of Example 8 can optionally include the lock comprising one or more of a holder or a clip.

In Example 10, the subject matter of any one or more of Examples 1-9 can optionally include the end effector comprising a jaw element.

In Example 11, the subject matter of any one or more of Examples 1-10 can optionally include a handpiece, operably connected to the end effector. The handpiece can be attached to and can extend from the proximal portion of the longitudinal shaft. The handpiece can be actuatable for operating the end effector.

Example 12 can include, or can be combined with the subject matter of any of Examples 1-11 to include a medical device. The medical device can comprise a longitudinal shaft, having a proximal portion and a distal portion. An end effector can be attached to and can extend from the distal portion. A handpiece can be operably connected to the end effector. The handpiece can be attached to and can extend from the proximal portion of the longitudinal shaft. A variable motion transfer assembly can be actuatable for adjusting force effected by the end effector.

In Example 13, the subject matter of any of Examples 1-12 can optionally include the variable motion transfer assembly comprising a compressible member, aligned with the longitudinal shaft. The compressible member can be configured for applying a variable maximum bias force for communication to the end effector. An end-user-positionable seat can be located against a first end of the compressible member. The seat can be actuatable by an end-user, such as for varying the variable maximum bias force.

In Example 14, the subject matter of any one or more of Examples 1-13 can optionally include the variable motion transfer assembly being actuatable for applying a continuously changeable force.

In Example 15, the subject matter of any one or more of Examples 1-14 can optionally include the variable motion transfer assembly being actuatable for applying a stepped changeable force.

Example 16 can include, or can be combined with the subject matter of one or more of Examples 1-15, to provide a method. The method can comprise: applying a first force to tissue with a forceps in a first mode of operation. The forceps can then be switched from the first mode of operation to a second mode of operation. The second mode of operation can comprise moving a user-positionable seat to compress a compressible member within the forceps such that the forceps are actuatable for applying a second force greater than the first force.

In Example 17, the subject matter of any of Examples 1-16 can optionally include applying the second force to tissue with the forceps.

In Example 18, the subject matter of any one or more of Examples 1-17 can optionally include switching the surgical forceps comprising moving the user-positionable seat with a slider and a lock.

In Example 19, the subject matter of any one or more of Examples 1-18 can optionally include switching the surgical forceps comprising changing a cam from a first position to a second position.

In Example 20, the subject matter of any one or more of Examples 1-19 can optionally include switching the surgical forceps back to the first mode of operation after using the surgical forceps in the second mode of operation.

Each of these non-limiting examples can stand on its own, or can be combined in various permutations or combinations with one or more aspects of one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. § 1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A medical device comprising:
a longitudinal shaft, having a proximal portion and a distal portion, with an end effector attached to and extending from the distal portion;
a compressible member, aligned with the longitudinal shaft, the compressible member configured for applying a variable maximum bias force for communication to the end effector; and
an end-user-positionable seat, located against a first end of the compressible member, the seat actuatable by an end-user for varying the variable maximum bias force.

2. The medical device of claim 1, wherein the compressible member comprises one or more of a spring, a coiled spring, a leaf spring, a disk spring, a helical spring, an overtravel spring, a double spring, or a pneumatic element.

3. The medical device of claim 1, wherein the end-user-positionable seat is actuatable for providing a continuous change to the bias force.

4. The medical device of claim 1, wherein the end-user-positionable seat is actuatable for providing a stepped change to the bias force.

5. The medical device of claim 1, wherein the end-user-positionable seat comprises a knob actuatable for rotating and moving the end-user positionable seat.

6. The medical device of claim 1, wherein the end-user-positionable seat comprises one or more of a nut, a hexagonal nut, a washer, a wedge, or a cam.

7. The medical device of claim 1, further comprising a hub at least partially surrounding the end-user-positionable seat and the compressible member.

8. The medical device of claim 1, wherein the end-user-positionable seat comprises a slider aligning with at least a portion of the longitudinal shaft, the slider moveable to release or secure the compressible member, and
a lock, actuatable by an end-user, the lock including end-user-selectable first and second states for respectively inhibiting or allowing the varying of the variable maximum bias force.

9. The medical device of claim 8, wherein the lock comprises one or more of a holder or a clip.

10. The medical device of claim 1, wherein the end effector comprises a jaw element.

11. The medical device of claim 1, further comprising a handpiece, operably connected to the end effector, the handpiece attached to and extending from the proximal portion of the longitudinal shaft, wherein the handpiece is actuatable for operating the end effector.

12. A medical device comprising:
a longitudinal shaft, having a proximal portion and a distal portion, with an end effector attached to and extending from the distal portion;
a handpiece, operably connected to the end effector, the handpiece attached to and extending from the proximal portion of the longitudinal shaft; and
a variable motion transfer assembly actuatable for adjusting force effected by the end effector.

13. The medical device of claim 12, wherein the variable motion transfer assembly comprises:
a compressible member, aligned with the longitudinal shaft, the compressible member configured for applying a variable maximum bias force for communication to the end effector; and
an end-user-positionable seat, located against a first end of the compressible member, the seat actuatable by an end-user for varying the variable maximum bias force.

14. The medical device of claim 12, wherein the variable motion transfer assembly is actuatable for applying a continuously changeable force.

15. The medical device of claim 12, wherein the variable motion transfer assembly is actuatable for applying a stepped changeable force.

16. A computer-implemented method comprising:
receiving user input to apply a first force to a target with a forceps with the forceps in a first mode of operation; and
switching the forceps from the first mode of operation to a second mode of operation at a user-specified maximum force level established by a user moving a user-positionable seat to compress a compressible member within the forceps such that the forceps are actuatable for applying a second force greater than the first force.

17. The method of claim 16, further comprising applying the second force to the target with the forceps.

18. The method of claim 16, further comprising moving the user-positionable seat with a slider and a lock to establish a force level at which the device switches from the first mode of operation to the second mode of operation.

19. The method of claim 16, wherein switching the forceps comprises changing a cam from a first position to a second position.

20. The method of claim 16, further comprising switching the forceps back to the first mode of operation after using the surgical forceps in the second mode of operation.
